(19) United States
(12) Patent Application Publication
(10) Pub. No.: US 2007/0196289 A1
(43) Pub. Date: Aug. 23, 2007

(54) Use Of Licochalcone A Or Of An Extract Of Licochalcone A From Radix Glycyrrhizae Inflatae Against Skin Aging

(75) Inventors: Thomas Blatt, Wedel (DE); Ute Breitenbach, Hamburg (DE); Stefan Gallinat, Wedel (DE); Ludger Kolbe, Dohren (DE); Christopher Mummert, Bienenbuettel (DE); Franz Staeb, Echem (DE); Rainer Wolber, Hamburg (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 10/571,530

(22) PCT Filed: Aug. 16, 2004
(86) PCT No.: PCT/EP04/09158

§ 371 Date: Nov. 30, 2006

(30) Foreign Application Priority Data
Sep. 12, 2003 (DE) 103 42 212.9

(51) Int. Cl. A61K 8/97; A61K 36/48

(52) U.S. Cl. 424/59; 424/74; 424/757

(57) ABSTRACT

Use of licochalcone A or of an extract containing licochalcone A from Radix Glycyrrhizae inflatae in cosmetic and dermatological preparations for the treatment and prophylaxis of the symptoms of intrinsic and/or extrinsic skin ageing and the treatment and prophylaxis of the damaging effects of ultraviolet radiation on the skin.

The present invention relates to cosmetic or dermatological preparations containing active ingredients for the care and protection of skin, particularly skin that is ageing or aged due to intrinsic and/or extrinsic factors, and to the use of such active ingredients and combinations of such active ingredients in the field of cosmetic and dermatological skin care.

Cosmetic skin care means primarily that the natural function of the skin becomes strengthened or restored as a barrier against environmental influences (for example, dirt, chemicals, microorganisms) and against the loss of endogenous materials (for example, water, natural oils, electrolytes).

If this function is disturbed, it can lead to intensified resorption of toxic or allergenic ingredients or to attack by microorganisms and consequently, to toxic or allergic skin reactions.

Furthermore, the objective of skin care is to compensate the skin for oil and water loss caused by daily washing. This is directly important if the natural regeneration capability does not suffice. In addition, skin care products should protect against environmental influences, especially sun and wind, and should delay skin ageing.

Chronological skin ageing is caused by, for example, endogenous, genetically determined factors. In the epidermis and dermis, ageing causes, for example, the following structural damage and function disorders, which can also be encompasses by the concept of “senile xerosis”:

a) dryness, roughness, and formation of dryness wrinkles,

b) itching, and

c) reduced oil restoration by sebaceous glands (for example, after washing).

Exogenous factors, such as UV light and chemically noxious agents, can take effect cumulatively, and for example, accelerate or supplement endogenous ageing processes. In the epidermis and dermis, exogenous factors result particularly, for example, in the following structural damage and disorders in the skin, which exceed qualitatively and quantitatively the damage of chronological ageing:

d) visible vasodilatations (telangiectasias, cuperosis);

e) flabbiness and formation of wrinkles;

f) local hyperpigmentation, hypopigmentation, and absence of pigmentation (for example, age spots), and

g) increased susceptibility to mechanical stress (for example, chapping).

The present invention involves, in particular, products for the care of naturally aged skin and the treatment of damage resulting from ageing by light, in particular, treatment of the phenomena listed under a) to g).

Products for the care of aged skin are known as such. They contain, for example, retinoids (vitamin A acid and/or its derivatives) or vitamin A and/or its derivatives. Their effect on structural damage is nevertheless moderately limited. Furthermore, there are significant difficulties in product development to stabilize the active ingredients adequately against oxidative decomposition. Furthermore, the use of products containing vitamin A acid often causes highly erythematic skin irritations. Therefore, retinoids are useful only in low concentrations.

In particular, the present invention relates to cosmetic preparations having effective protection against damaging oxidation processes in the skin, but also for the protection of cosmetic preparations themselves or for the protection of components of cosmetic preparations from damaging oxidative processes.

The present invention furthermore relates to antioxidants, preferably those used in cosmetic or dermatological skin care preparations. In particular, the invention also relates to cosmetic and dermatological preparations containing such antioxidants.

In a preferred embodiment, the present invention relates to cosmetic and dermatological preparations for the prophylaxis and treatment of cosmetic or dermatological skin changes, such as, for example, skin ageing, particularly skin ageing caused by oxidative processes.

In a further advantageous embodiment, the present invention relates to combinations of active ingredients and preparations that serve the prophylaxis and treatment of light-sensitive skin, in particular, photodermatoses.

The damaging effect of the ultraviolet region of sun radiation on skin is generally known. While rays having a wavelength smaller than 290 nm (the so-called UVC range) are absorbed by the ozone layer in the earth's atmosphere, rays in the range between 290 nm and 320 nm, the so-called UVB range, cause erythema, a simple sunburn, or even more or less intense burns.

The narrower range at around 308 nm is stated as a maximum for the erythema effect of sunlight.

Many compounds are known for protection against UVB radiation, among them being derivatives of 3-benzylidene camphor, of 4-aminobenzoic acid, of cinnamic acid, of salicylic acid, of benzophenone, as well as of 2-phenylbenzimidazole.

It is also important to have filter substances available for the range between about 320 nm and about 400 nm, the so-called UVA range, because its rays can cause reactions in light-sensitive skin. It has been shown that UVA radiation leads to damage in the elastic and collagenic fibers of the connective tissue, which ages skin prematurely, and that is seen as the cause of many phototoxic and photoallergenic reactions. The damaging effect of UVB radiation can be intensified by UVA radiation. Therefore, to protect against radiation in the UVA range, certain derivatives of dibenzoyl methane are used, the stability of which is stated as inadequate (Int. J. Cosm. Science 10, 53 (1988)).

However, UV radiation can also lead to photochemical reactions, whereby the photochemical reaction products attack the skin metabolism.

Such photochemical reaction products are predominantly radical compounds, for example, hydroxy radicals. Also, undefined radical photoproducts, which develop spontaneously in the skin, can exhibit uncontrolled subsequent reactions due to their high reactivity. However, singlet oxygen, a non-radical excited state of the oxygen molecule, can occur from UV radiation, as well as short-lived epoxides and many others. Singlet oxygen, for example, is characterized by increased reactivity compared to the normally present triplet oxygen (radical ground state). However, excited, reactive (radical) triplet states of the oxygen molecule also exist.

Moreover, UV radiation ranks with ionizing radiation. Thus, here exists the risk that also ionic species develop on UV exposure, which then can engage oxidatively in biochemical processes.

To prevent these reactions, additional antioxidants and/or free-radical scavengers can be incorporated in cosmetic or dermatological formulations.

Vitamin E, a substance of known antioxidant activity, has already been proposed for use in photoprotective formulations; yet the desired effect here is far from that hoped for.

Therefore, the object of the invention also was to provide cosmetic, dermatological and pharmaceutical active ingredients and preparations, as well as photoprotective formulations, which serve for the prophylaxis and treatment of light sensitive skin, in particular photodermatoses, preferably PLD.

Other designations for polymorphic light dermatosis are PLD, PLE, mallorca acne, and many other designations, as stated in the literature (for example, A. Voelckel et al, Zentralblatt Haut- und Geschlechtskrankheiten (1989), 156, page 2).

Erythematic skin phenomena also occur as concomitant phenomena in certain skin ailments or skin irregularities. For example, the typical skin rash in the occurrence of acne regularly is of more or less redness.

Antioxidants are used mainly to protect against spoilage of preparations containing them. Yet, it is known that undesired oxidation processes can also occur in human and animal skin. Such processes play a substantial role in skin-ageing.

Oxidative skin damage and its deeper causes are discussed in the article “Skin Diseases Associated with Oxidative Injury” in “Oxidative Stress in Dermatology”, pages 323 ff (Marcel Decker Inc. New York, Basel, Hong Kong, editors: Jürgen Fuchs, Frankfurt, and Lester Packer, Berkeley, Calif.).

Also on the grounds of preventing such reactions, additional antioxidants and/or radical scavengers may be incorporated in cosmetic or dermatological formulations.

Indeed, several antioxidants and free radical scavengers are known. Thus, U.S. Pat. Nos. 4,144,325 and 4,428,861 and a number of other documents have already proposed using vitamin E, a substance of known antioxidant effect, in formulations to protect against light; however, here too the desired effect is far from that hoped for.

Consequently, the object of the present invention was to find ways of avoiding the disadvantages of the prior art. In particular, the effect of repairing damage associated with endogenous, chronological, and exogenous skin ageing and the prophylaxis should be durable, lasting, and without the risk of side effects.

The object of the present invention was to remedy these drawbacks.

It has surprisingly been found that the use of licochalcone A or of an extract containing licochalcone A from Radix Glycyrrhizae inflatae in cosmetic or dermatological preparations for the treatment and prophylaxis of symptoms of intrinsic and/or extrinsic skin ageing as well as for the treatment and prophylaxis of the damaging effects of ultraviolet radiation on the skin remedies the disadvantages of the prior art.

The application of the active ingredient according to the present invention or of cosmetic or topical dermatological preparations having an effective content of the active ingredient according to the present invention surprisingly enables an effective treatment, but also a prophylaxis
of deficient or hypoactive skin conditions or deficient or hypoactive states of skin appendages,
of phenomena of premature ageing of skin (for example, wrinkles, age spots, telangiectasias) and/or of skin appendages,
of negative changes in the skin and skin appendages caused by the environment (smoking, smog, reactive oxygen species, free radicals),
of skin damage caused by light,
of itching
of dry skin conditions and horny layer barrier disorders,
of hair loss and for improved hair growth,
of inflammatory skin conditions, such as seborrheic eczema, polymorphic light dermatosis and vitiligo.

The active ingredient of the present invention or cosmetic or topical dermatological preparations having an effective content of the active ingredient of the present invention surprisingly also serve
for stimulation of collagen synthesis, hyaluronic acid synthesis, elastin synthesis,
for stimulation of intracellular DNA synthesis, especially in deficient or hypoactive skin conditions,
for increasing cell renewal and skin regeneration,
for increasing the skin's inherent protective and repair mechanisms (for example, for disfunctional enzymes, DNA, lipids, proteins),
for pretreatment and post-treatment in the topical use of laser treatments and removal treatments, which serve, for example, to reduce skin wrinkles and scars, to counteract the resulting skin irritations and to promote regeneration processes in the injured skin.

It is, in particular, extremely advantageous according to the present invention to use the active ingredient of the present invention or cosmetic or topical dermatological preparations having an effective content of the active ingredient of the present invention for the cosmetic or dermatological treatment or prophylaxis of undesired skin conditions.

Especially advantageous is a use according to the present invention which is characterized in that the preparations contain from 0.0001% to 5% by weight, particularly from 0.001% to 1% by weight, most particularly from 0.005% to 0.15% by weight of licochalcone A, relative to the total weight of the preparation.

Further particularly advantageous is a use according to the present invention which is characterized in that the preparations contain from 0.001% to 10% by weight, particularly from 0.05% to 5% by weight, most particularly from 0.01% to 2% by weight of one or more polyols, relative to the total weight of the preparation.

Further particularly advantageous is a use according to the present invention which is characterized in that the preparations contain licochalcone as a component from plant extracts, particularly from Radix Glycyrrhizae inflatae.

Like the licorice Glycyrrhizae glabra which is official in Europe, the plant type Glycyrrhizae inflatae belongs to the genus Glycyrrhiza, which belongs to the Fabacae plant family (pea plants). The drug Radix Glycyrrhizae inflatae, that is, the root of the plant, is commonly used in, for example, Far Eastern medicine. The use of the drug as antiinflammatory agent is similarly known.

A component of the aqueous extract from Radix Glycyrrhizae inflatae is licochalcone A, which is characterized by the following structural formula:

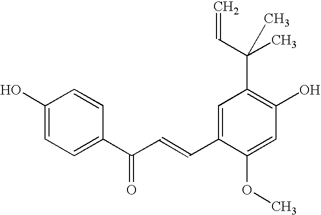

It is assumed that this substance participates in the effect according to the present invention, possibly synergistically with the remaining components of the extract.

Accordingly, also according to the present invention is the use of a combination of
licochalcone A,
water,
optionally, one or more polyols
   in cosmetic or dermatological preparations for the treatment and prophylaxis of the symptoms of intrinsic and/or extrinsic skin ageing and for the treatment and prophylaxis of the damaging effects of ultraviolet radiation on skin.

It is advantageous, according to the invention, if the cosmetic or dermatological preparations contain from 0.001% to 10% by weight, particularly from 0.05% to 5% by weight, most particularly from 0.01% to 2% by weight of an aqueous extract from Radix Glycyrrhizae inflatae, relative to the total weight of the preparation.

It is advantageous, according to the invention, if the cosmetic or dermatological preparations contain from 0.001% to 10% by weight, particularly from 0.05% to 5% by weight, most particularly from 0.01% to 2% by weight of one or more ethoxylated or propoxylated raw materials, relative to the total weight of the preparation.

It is advantageous, according to the present invention, if the cosmetic or dermatological preparations contain from 0.001% to 10% by weight, particularly from 0.05% to 5% by weight, most particularly from 0.01% to 2% by weight of one or more polyols, relative to the total weight of the preparation.

Particularly, it is advantageous to select butylene glycol as the polyol.

It is most particularly advantageous to start from an extract that is marketed by Maruzen Company under the designation Polyol Soluble Licorice Extract P-U.

Furthermore, it is advantageous to use licochalcone A in other vehicle systems in a concentration of from 0.0001% to 5% by weight, particularly from 0.001% to 1% by weight, most particularly 0.005-0.05% by weight.

In accordance with the present invention, preparations that contain combinations of active ingredients of the present invention can use conventional antioxidants.

The antioxidants are advantageously selected from the group consisting of amino acids (for example, glycine, histidine, tyrosine, tryptophane) and their derivatives, imidazoles (for example, urocaninic acid) and their derivatives, peptides, such as D,L-carnosine, D-carnosine, L-carnosine and their derivatives (for example, anserine) carotinoids, carotines (for example, α-carotene, β-carotene, lycopene) and their derivatives, lipoic acid and its derivatives (for example, dihydrolipoic acid, aurothioglucose, propyl thiouracil and other thiols (for example, thioredoxin, glutathione, cysteine, cystine, cystamine, and their glycosyl, N-acetyl, methyl, ethyl, propyl, amyl, butyl and lauryl, palmitoyl, oleyl, γ-linoleyl, cholesteryl, and glyceryl esters) and their salts, dilauryl thiodipropionate, distearyl thiodipropionate, thiodipropionic acid and their derivatives (esters, ethers, peptides, lipids, nucleotides, nucleosides, and salts), as well as sulfoximine compounds (for example, buthionine sulfoximines, homocysteine sulfoximine, buthionine sulfones, penta-, hexa-, heptathionine sulfoxime) in very small compatible doses (for example, pmole to μmole/kg), further, (metal) chelating agents (for example, α-hydroxy fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA, and their derivatives, unsaturated fatty acids and their derivatives (for example, γ-linolenic acid, linoleic acid, oleic acid), folic acid and its derivatives, alanine diacetic acid, flavonoids, polyphenols, catechins, vitamin C and derivatives (for example, ascorbyl palmitate, Mg-ascorbyl phosphate, ascorbyl acetate), tocopheroles and derivatives (for example, vitamin E acetate), and coniferyl benzoate of benzoin resin, rutinic acid and its derivatives, ferulic acid and its derivatives, butylated hydroxytoluene, butylated hydroxyanisole, nordihydroguaiac resin acid, nordihydroguajaretic acid, trihydroxy butyrophenone, uric acid and its derivatives, mannose and its derivatives, zinc and its derivatives (for example, ZnO, ZnSO₄), selenium and its derivatives (for example, selenium methionine), stilbene and its derivatives (for example, stilbene oxide, trans-stilbene oxide), and derivatives of these named ingredients (salts, esters, ethers, sugars, nucleotides, nucleosides, peptides, and lipids) which are suitable according to the present invention.

The amount of antioxidants (one or more compounds) in the preparations is preferably from 0.001% to 30% by weight, especially preferred 0.05-20% by weight, particularly 1-10% by weight, relative to the total weight of the preparation.

The prophylaxis or the cosmetic or dermatological treatment with the active ingredient used according to the present invention or with cosmetic or topical dermatological preparations having an effective content of the active ingredient used according to the present invention takes place in the usual manner, that is, in such a manner that the active ingredient used according to the present invention or the cosmetic or topical dermatological preparations having an effective content of the active ingredient used according to the present invention are applied on the affected skin areas.

The active ingredient used according to the present invention can advantageously be incorporated in conventional cosmetic and dermatological preparations, which can be present in various forms. Thus, these can be, for example, a solution, an emulsion of the water-in-oil type (W/O) or the oil-in-water type (O/W), or a multiple emulsion, for example, of the water-in-oil-in-water type (W/O/W), a hydrodispersion or lipodispersion, a gel, a solid stick, or also an aerosol

Emulsions according to the present invention in the sense of the present invention, for example, in the form of a cream, a lotion, a cosmetic milk are advantageous and contain, for example, fats, oils, waxes and/or other fatty structures, as well as water and one or more emulsifiers, such as are used conventionally for such a type of formulation.

It is also possible and advantageous in the sense of the present invention to incorporate the active ingredient used according to the present invention in aqueous systems or surfactant preparations for cleansing skin and hair.

One of skill in the art naturally knows that exacting cosmetic compositions are mostly not conceivable without the conventional auxiliaries and additives. Examples among these are builders, fillers, perfume, dyes, emulsifiers, additional ingredients, such as vitamins or proteins, photoprotective agents, stabilizers, insect repellents, alcohol, water, salts, antimicrobials, proteolytically or keratolytically effective substances, etc.

With the necessary changes, corresponding requirements apply to the formulation of medical preparations.

Topical medical compositions in the sense of the present invention contain as a rule one or more medications in effective concentration. For the sake of simplicity, reference is made for the clear-cut difference between cosmetic and medical use and corresponding products to the legal provisions of the Federal Republic of Germany (for example, cosmetic ordinance, foodstuff law and drug law).

It is similarly advantageous to add the active ingredient used according to the present invention to preparations that already contain other active ingredients for other purposes.

Accordingly, cosmetic or topical dermatological compositions in the sense of the present invention, depending on their makeup, can be used, for example, as skin protection cream, cleansing milk, sunscreen lotion, nutrient cream, day or night cream, etc. It is optionally possible and advantageous to use the compositions according to the present invention as a base for pharmaceutical formulations.

Cosmetic and dermatological preparations optionally present in the form of a sunscreen agent are also favorable. These contain preferably, in addition to the active ingredient used according to the present invention, at least one UVA filter substance and/or at least one UVB filter substance and/or at least one inorganic pigment.

But it is also advantageous in the sense of the present invention to make cosmetic and dermatological preparations, the main purpose of which is not protection from sunlight, but which, yet, contain UV protection substances. Thus, for example, conventional UVA or UVB filter substances are usually incorporated in a day cream.

The preparations according to the present invention can advantageously contain substances that absorb UV radiation in the UVB range, whereby the total quantity of the filter substances is, for example, from 0.1% by weight to 30% by weight, preferably from 0.5% to 10% by weight, particularly from 1% to 6% by weight, relative to the total weight of the preparations.

The UVB filters can be oil-soluble or water-soluble. Examples of oil-soluble substances are:
3-benzylidenecamphor and its derivatives, for example, 3-(4-methyl-benzylidene)camphor,
4-aminobenzoic acid derivatives, preferably 2-ethylhexyl 4-(dimethylamino)-benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone;
esters of benzalmalonic acid, preferably di(2-ethylhexyl) 4-methoxy-benzalmalonate;
2,4,6-trianilino-(p-carbo-2′-ethyl-1′-hexyloxy)-1,3,5-triazine.

Advantageous as water-soluble substances are:
2-phenylbenzimidazole-5-sulfonic acid and its salts, for example, sodium, potassium, or triethanol ammonium salts;
sulfonic acid derivatives of benzophenones, preferably 2-hydroxy-4-methoxy-benzophenone-5-sulfonic acid and its salts;
sulfonic acid derivatives of 3-benzylidenecamphor, such as, for example, 4-(2-oxo-3-bornylidenemethyl)benzene sulfonic acid, 2-methyl-5-(2-oxo-3-bornylidene-methyl)sulfonic acid and their salts.

The lists of the named UVB filters that can be used in the invention is, of course, not intended to be limiting.

Also an object of the invention is the combination of a UVA filter according to the invention with a UVB filter or a cosmetic or dermatological preparation according to the invention, which also contains a UVB filter.

It can also be advantageous to incorporate in the preparations according to the present invention UVA filters that are usually contained in cosmetic and/or dermatological preparations. Such filter substances preferably are derivatives of dibenzoyl methane, particularly 1-(4′-t-butylphenyl)-3-(4′-methoxyphenyl)propane-1,3-dione and 1-phenyl-3-(4′-isopropyl-phenyl)propane-1,3-dione. Preparations containing these combinations are also an object of the invention. The same quantities of UVA filter substances can be used as were given for UVB filter substances.

Cosmetic and/or dermatological preparations in the sense of the present invention can also contain inorganic pigments that are conventionally used in cosmetics to protect the skin from UV radiation. These involve oxides of titanium, zinc, iron, zirconium, silicon, manganese, aluminum, cerium and mixtures thereof, as well as variations wherein the oxides are the active agents. Especially preferred are pigments on the basis of titanium dioxide. Quantities stated for the preceding combinations can be used.

The cosmetic and dermatological preparations according to the present invention can contain cosmetic active ingredients, auxiliaries and/or additives as are conventionally used in such preparations, for example, antioxidants, preservatives, bactericides, perfumes, foam inhibitors, dyes, coloring pigments, thickeners, surfactants, emulsifiers, softeners, moisturizers and/or humectants, fats, oils, waxes or other usual components of a cosmetic or dermatological formulation, such as alcohols, polyols, polymers, foam stabilizers, electrolytes, organic solvents or silicone derivatives.

Insofar as the cosmetic or dermatological preparation in the sense of the present invention is a solution or emulsion or dispersion, solvents that can be used are:
water or aqueous solutions;
oils, such as triglycerides of capric acid or caprylic acid, but preferably castor oil;
fats, waxes, and other natural and synthetic fat structures, preferably esters of fatty acids with alcohols of low carbon number, for example, with isopropanol, propylene glycol, or glycerin, or esters of fatty alcohols with alkanoic acids of low carbon number or with fatty acids;
alcohols, diols or polyols of low carbon number, and their ethers, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the preceding solvents are used. Water can be an additional component with alcoholic solvents.

The oil phase of the emulsions, oleogels or hydrodispersions or lipodispersions in the sense of the present invention are advantageously selected from group of esters of saturated and/or unsaturated, branched and/or unbranched alkanecarboxylic acids of a chain length of from 3 to 30 carbon atoms and saturated and/or unsaturated, branched and/or unbranched alcohols of a chain length of from 3 to 30 carbon atoms, from the group of esters of aromatic carboxylic acids and saturated and/or unsaturated, branched and/or unbranched alcohols of a chain length of from 3 to 30 carbon atoms. Such ester oils can then be advantageously selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic semi-synthetic, and natural mixtures of such esters, for example, jojoba oil.

Furthermore, the oil phase can be selected from the group of branched and linear hydrocarbons and hydrocarbon waxes, silicone oils, dialkyl ethers, the group of saturated or unsaturated, branched or linear alcohols, and fatty acid triglycerides, specifically triglycerin esters of saturated and/or unsaturated, branched and/or unbranched alkanecarboxylic acids of a chain length of from 8 to 24, particularly 12-18 carbon atoms. The fatty acid triglycerides can be selected advantageously, for example, from the group of synthetic, semi-synthetic, and natural oils, for example, olive oil, sunflower oil, soy oil, peanut oil, rapeseed oil, almond oil, palm oil, coconut oil, palm kernel oil, and more of the like.

Any blends of such oil and wax components can advantageously be used in the sense of the present invention. It can optionally also be advantageous to incorporate waxes, for example, cetyl palmitate, as the only lipid component of the oil phase.

The oil phase is advantageously selected from the group 2-ethylhexyl isostearate, octyl dodecanol, isotridecyl isononanoate, isoeicosane, 2-ethylhexyl cocoate, C₁₂₋₁₅ alkyl benzoate, caprylic-capric acid triglyceride, dicaprylyl ether.

Especially advantageous are mixtures of C₁₂₋₁₅ alkyl benzoate and 2-ethylhexyl isostearate, mixtures of C₁₂₋₁₅ alkyl benzoate and isotridecyl nonanoate, and mixtures of C₁₂₋₁₅ alkyl benzoate, 2-ethylhexyl isostearate, and isotridecyl isononanoate.

Paraffin oil, squalane, and squalene, among the hydrocarbons, can be used advantageously in the sense of the present invention.

Moreover, the oil phase can advantageously have a content of cyclic or linear silicone oils or consist completely of such oils, it, nevertheless, being preferred to use an additional content of other oil phase components, apart from the silicone oil or silicone oils.

Cyclomethicone (octamethyl cyclotetrasiloxane) is advantageously used as the silicone oil for use in the present invention. However, other silicone oils are advantageous to use in the sense of the invention, for example, hexamethyl cyclotrisiloxane, polydimethyl siloxane, poly(methylphenylsiloxane).

Especially advantageous further are mixtures of cyclomethicone and isotridecyl nonanoate, and of cyclomethicone and 2-ethylhexyl isostearate.

The aqueous phase of the preparations according to the present invention optionally contains advantageously:

alcohols, diols, or polyols of low carbon number and their ethers, preferably ethanol, isopropanol, propylene glycol, glycerin, ethylene glycol, ethylene glycol monoethyl or monobutyl ether, propylene glycol monomethyl, monoethyl, or monobutyl ether, diethylene glycol monomethyl or monoethyl ether and analogous products, further alcohols of low carbon number, for example, ethanol, isopropanol, 1,2-propanediol, glycerin, and particularly, one or more thickening agents, which can advantageously be selected from the group silicon dioxide, aluminum silicate, polysaccharides or their derivatives, for example, hyaluronic acid, xanthan gum, hydroxypropyl methylcellulose, especially advantageously from the group of polyacrylates, preferably a polyacrylate from the group of so-called Carbopols, for example, Carbopols of Types 980, 981, 1382, 2984, 5984, in each case, alone or in combination.

Gels used according to the invention usually contain alcohols of low carbon number. for example, ethanol, isopropanol, 1,2-propanediol, glycerin and water or a previously named oil in the presence of a thickening agent, that is preferably silicon dioxide or an aluminum silicate in oily-alcoholic gels and preferably a polyacrylate, in aqueous alcoholic or alcoholic gels.

Solid sticks contain, for example, natural or synthetic waxes, fatty alcohols or fatty acid esters.

Conventional base materials, which are suited for use as cosmetic sticks in the sense of the present invention, are liquid oils (for example, paraffin oils, castor oil, isopropyl myristate), semi-solid components (for example, petrolatum, lanolin), solid components (for example, beeswax, ceresin and microcrystalline waxes or ozokerite) and high-melting waxes (for example, carnauba wax, candelilla wax).

As propellants for cosmetic and/or dermatological preparations in the sense of the present invention, sprayable from aerosol containers, are suitable the usual known, easily volatile, liquified propellants, for example, hydrocarbons (propane, butane, isobutane), which can be use individually or in mixtures thereof. Compressed air is also advantageous to use.

One of skill in the art naturally knows that there are nontoxic propellant gases, which in principle would be suited as aerosol preparations of the present invention, but which nevertheless should be dispensed with due to their serious effect on the environment or other associated circumstances, particularly fluorocarbons and fluorochlorocarbons (FCKW).

Cosmetic preparations in the sense of the present invention can also be present as gels that contain in addition to an effective content of the active ingredient according to the present invention and solvents conventionally used for it, preferably water, also organic thickeners, for example, gum arabic, xanthan gum, sodium alginate, cellulose derivatives, preferably methyl cellulose, hydroxymethyl cellulose, hydroxyethyl cellulose, hydroxypropyl cellulose, hydroxypropylmethyl cellulose, or inorganic thickeners, for example, aluminum silicates, such as, for example, bentonites or a mixture of polyethylene glycol and polyethylene glycol stearate or distearate. The quantity of the thickening agent in the gel is, for example, between 0.1% and 30% by weight, preferably between 0.5% and 15% by weight.

The following examples are to illustrate the present invention without limiting it. All quantity data, parts and percentages are based, unless otherwise stated, on the weight and the total quantity or the total weight of the preparations.

### EXAMPLES OF O/W CREAMS

Example No. 1

| | |
|---|---|
| Glyceryl stearate, self-emulsifying | 4.00 |
| PEG-40 stearate | 1.00 |
| Cetyl alcohol | 3.00 |
| Caprylic-/capric triglyceride | 5.00 |
| Mineral oil | 5.00 |
| Polyol Soluble Licorice Extract P-U | 0.025 |
| Tocopherol | 0.1 |
| Na₂HEDTA | 0.1 |
| Preservative(s), perfume | q.s. |
| Polyacrylic acid | 3.00 |
| Sodium hydroxide solution 45% | q.s. |
| Glycerin | 5.00 |
| Water | to 100 |

Example No. 2

| | |
|---|---|
| Glyceryl stearate, self-emulsifying | 3.00 |
| Stearic acid | 1.00 |
| Cetyl alcohol | 2.00 |
| Caprylic-/capric triglyceride | \3.00 |
| Dicaprylyl ether | 4.00 |
| Mineral oil | 2.00 |
| Polyol Soluble Licorice Extract P-U | 0.05 |
| Preservative(s), perfume | q.s. |
| Polyacrylic acid | 0.1 |
| Sodium hydroxide solution 45% | q.s. |
| Glycerin | 3.00 |
| Butylene glycol | 3.00 |
| Water | ad 100 |

Example No. 3

| | |
|---|---|
| Glyceryl stearate citrate | 2.00 |
| Stearyl alcohol | 2.00 |
| Lanolin alcohol | 1.00 |
| Caprylic-/capric triglyceride | 4.00 |
| Mineral oil | 8.00 |
| Dimethicone | 1.00 |
| Licochalcone A | 0.025 |
| Preservative(s), perfume | q.s. |
| Sodium hydroxide solution 45% | q.s. |
| Glycerin | 7.50 |
| Water | ad 100 |

Example No. 4

| | |
|---|---|
| Glyceryl stearate citrate | 2.00 |
| Stearyl alcohol | 2.00 |
| Lanolin alcohol | 1.00 |
| Caprylic-/capric triglyceride | 4.00 |
| Mineral oil | 8.00 |
| Dimethicone | 1.00 |
| Polyol Soluble Licorice Extract P-U | 0.15 |
| Preservative(s), perfume | q.s. |
| Sodium hydroxide solution 45% | q.s. |
| Glycerin | 7.50 |
| Dihydroxy acetone | 1.00 |
| Water | ad 100 |

Example No. 5

| | |
|---|---|
| Polyglyceryl-3-methylglucose distearate | 3.00 |
| Cetyl alcohol | 3.00 |
| Caprylic-/capric triglyceride | 3.00 |
| Dicaprylyl ether | 2.00 |
| Mineral oil | 3.00 |
| Polyol Soluble Licorice Extract P-U | 1.00 |
| Na3HEDTA | 0.1 |
| Preservative(s), perfume | q.s. |
| Polyacrylic acid | 0.1 |
| Sodium Hydroxide solution 45% | q.s. |
| Glycerin | 3.00 |
| Water | ad 100 |

Example No. 6

| | |
|---|---|
| Glyceryl stearate citrate | 2.00 |
| Sorbitan stearate | 2.00 |
| Cetyl stearyl alcohol | 2.00 |
| Caprylic-/capric triglyceride | 3.00 |
| Octyl dodecanol | 2.00 |
| Dicaprylyl ether | 1.00 |
| Polyol Soluble Licorice Extract P-U | 0.05 |
| Tocopherol | 0.20 |
| Preservative(s), perfume | q.s. |
| Polyacrylic acid | 0.1 |
| Sodium hydroxide solution 45% | q.s. |
| Glycerin | 3.00 |
| Water | ad 100 |

### EXAMPLES OF O/W CREAMS

Example No. 7

| | |
|---|---|
| Glyceryl stearate, self-emulsifying | 5.00 |
| Stearyl alcohol | 2.00 |
| Caprylic-/capric triglyceride | 2.00 |
| Octyl dodecanol | 2.00 |
| Dimethicone polydimethylsiloxane | 2.00 |
| Titanium dioxide | 2.00 |
| 4-Methylbenzylidene camphor | 1.00 |
| Butylmethoxy dibenzoylmethane | 0.50 |
| Licochalcone A | 0.08 |
| Preservative(s), perfume | q.s. |
| Polyacrylic acid | 0.15 |
| Sodium hydroxide solution 45% | q.s. |
| Glycerin | 3.00 |
| Water | ad 100 |

Example No. 8

| | |
|---|---|
| Glyceryl stearate citrate | 2.00 |
| Cetyl stearyl alcohol | 3.00 |
| C₁₂₋₁₅ Alkyl benzoate | 2.00 |
| Octyl dodecanol | 2.00 |
| Mineral oil | 4.00 |
| Polyol Soluble Licorice Extract P-U | 0.50 |
| 2,4-Bis-(2-ethyl-hexyloxy-)2-hydroxyl)-phenyl)-6-(4- | 1.0 |
| methoxyphenyl)-(1,3,5)-triazine | |
| Dihydroxyacetone | 0.5 |
| Preservative(s), perfume | q.s. |
| Polyacrylic acid | 0.1 |
| Sodium hydroxide solution 45% | q.s. |
| Butylene glycol | 3.00 |
| Ethanol | 3.00 |
| Water | ad 100 |

Example No. 9

| | |
|---|---|
| Glyceryl stearate citrate | 2.00 |
| Cetyl stearyl alcohol | 1.00 |
| C₁₂₋₁₅ Alkyl benzoate | 3.00 |
| Mineral oil | 2.00 |
| Polyol Soluble Licorice Extract P-U | 0.1 |
| 2,4-bis-(2-ethyl-hexyloxy-)2-hydroxyl)-phenyl)-6-(4- | 3.0 |
| methoxyphenyl)-(1,3,5)-triazine | |
| Ethylenediamine tetraacetic acid, trisodium | 0.20 |
| Preservative(s), perfume | q.s. |
| Xanthan gum | 0.20 |
| Sodium hydroxide solution 45% | q.s. |
| Glycerin | 3.00 |
| Water | ad 100 |

Example No. 10

| | |
|---|---|
| Stearic acid | 2.50 |
| Cetyl alcohol | 3.00 |
| Octyl dodecanol | 4.00 |
| Cyclic dimethyl polysiloxane | 0.50 |
| Polyol Soluble Licorice Extract P-U | 1.00 |
| Preservative(s), perfume | q.s. |
| Polyacrylic acid | 0.05 |
| Sodium hydroxide solution 45% | q.s. |
| Glycerin | 5.00 |
| Ethanol | 3.00 |
| Water | ad 100 |

Example No. 11

| | |
|---|---|
| Stearic acid | 3.50 |
| Cetyl alcohol | 4.50 |
| Cetyl stearyl alcohol | 0.50 |
| Octyl dodecanol | 6.00 |
| Cyclic dimethyl polysiloxane | 2.00 |
| 4-Methyl benzylidene camphor | 1.00 |
| Butylmethoxy dibenzoylmethane | 0.50 |
| Polyol Soluble Licorice Extract P-U | 0.40 |
| 2,4-Bis-(2-ethyl-hexyloxy-)2-hydroxyl)-phenyl)-6-(4- | 0.5 |
| methoxyphenyl)-(1,3,5)-triazine | |
| Dihydroxyacetone | 0.5 |
| Tocopherol | 0.05 |
| Ethylenediamine tetraacetic acid, trisodium | 0.20 |
| Preservative(s), perfume | q.s. |
| Polyacrylic acid | 0.05 |
| Sodium hydroxide solution 45% | q.s. |
| Glycerin | 3.00 |

### EXAMPLE OF W/O EMULSIONS

Example No. 12

| | |
|---|---|
| Polyglyceryl-2-dipolyhydroxystearate | 5.00 |
| 2,4-Bis-(2-ethyl-hexyloxy-)2-hydroxyl)-phenyl)-6-(4- | 2.00 |
| methoxyphenyl)-(1,3,5)-triazine | |
| Diethylhexyl butamidotriazone | 3.00 |
| Octocrylene | 7.00 |
| Diethylhexyl butamidotriazone | 1.00 |
| Phenylene-1-4-bis-(monosodium,2-benzimidazyl-5,7- | 1.00 |
| disulfonic acid) | |
| Phenylbenzimidazole sulfonic acid | 0.50 |
| Zinc oxide | 3.00 |
| Dicaprylyl ether | 10.00 |
| Dicaprylyl carbonate | 5.00 |
| Phenylmethyl polysiloxane | 2.00 |
| PVP hexadecene copolymer | 0.50 |
| Glycerin | 3.00 |
| Magnesium sulfate | 1.00 |
| Tocopherol acetate | 0.50 |
| Polyol Soluble Licorice Extract P-U | 0.15 |
| Preservative(s), perfume | q.s. |
| Ethanol | 3.00 |
| Water | ad 100 |

Example No. 13

| | |
|---|---|
| Cetyl dimethicone copolyol | 2.50 |
| 2-Ethylhexyl methoxy cinnamate | 8.00 |
| 2,4-Bis-(4-(2-ethyl-hexyloxy-)2-hydroxyl)-phenyl)-6-(4- | 2.50 |
| methoxyphenyl)-(1,3,5)-triazine | |
| Diethylhexyl butamidotriazone | 1.00 |
| 4-methyl benzylidene camphor | 2.00 |
| Octocrylene | 2.50 |
| Phenylene-1,4-bis-(monosodium,2-benzimidazyl-5,7- | 2.00 |
| disulfonic acid) | |
| Titanium dioxide | 2.00 |
| Zinc oxide | 1.00 |
| Dimethicone polydimethylsiloxane | 4.00 |
| Phenylmethylpolysiloxane | 25.00 |
| Octoxyglycerin | 0.30 |
| Glycerin | 7.50 |
| Glycine soy | 1.00 |
| Magnesium sulfate | 0.50 |
| Polyol Soluble Licorice Extract P-U | 0.08 |
| Preservative(s), perfume | q.s. |
| Water | ad 100 |

Example No. 14

| | |
|---|---|
| PEG-30 dipolyhydroxystearate | 5.00 |
| Butylmethoxy dibenzoylmethane | 2.00 |
| Ethylhexyltriazone | 3.00 |
| Octocrylene | 4.00 |
| Phenylene-1,4-bis-(monosodium,2-benzimidazyl-5,7- | 0.50 |
| disulfonic acid) | |
| Titanium dioxide | 1.50 |
| Zinc oxide | 2.00 |
| Mineral oil | 10.0 |
| Butylene glycol dicaprylate/dicaprate | 2.00 |
| Dicaprylyl carbonate | 6.00 |
| Dimethicone polydimethyl siloxane | 1.00 |
| Shea butter | 3.00 |
| Octoxy glycerin | 1.00 |
| Glycine soy | 1.50 |
| Magnesium chloride | 1.00 |
| Tocopherol acetate | 0.25 |
| Polyol Soluble Licorice Extract P-U | 0.5 |
| Preservative(s), perfume | q.s. |
| Ethanol | 1.50 |
| Water | ad 100 |

Example No. 15

| | |
|---|---|
| Cetyl dimethicone copolyol | 4.00 |
| 2-Ethylhexyl methoxy cinnamate | 5.00 |
| 2,4-Bis-(2-ethyl-hexyloxy-)2-hydroxyl)-phenyl)-6-(4- | 2.00 |
| methoxyphenyl)-(1,3,5)-triazine | |
| Butylmethoxy dibenzoylmethane | 1.00 |
| Ethylhexyl triazone | 4.00 |
| 4-Methylbenzylidene camphor | 4.00 |
| Diethylhexyl butamidotriazone | 2.00 |
| Phenylbenzimidazole sulfonic acid | 3.00 |
| Zinc oxide | 0.50 |
| C₁₂₋₁₅ Alkyl benzoate | 9.00 |
| Butylene glycol dicaprylate/dicaprate | 8.00 |
| Dimethicone polydimethyl siloxane | 5.00 |
| PVP hexadecene copolymer | 0.50 |
| Glycerin | 7.50 |
| Magnesium sulfate | 0.50 |
| Polyol Soluble Licorice Extract P-U | 1.00 |
| Preservative(s), perfume | q.s. |
| Water | ad 100 |

Example No. 16

| | |
|---|---|
| Polyglyceryl-2-dipolyhydroxy stearate | 4.50 |
| 2-Ethylhexyl methoxy cinnamate | 4.00 |
| 2,4-Bis-(2-ethyl-hexyloxy-)2-hydroxyl)-phenyl)-6-(4- | 2.50 |
| methoxyphenyl)-(1,3,5)-triazine | |
| Diethylhexyl butamidotriazone | 3.00 |
| Ethylhexyl triazone | |
| 4-Methylbenzylidene camphor | 2.00 |
| Octocrylene | 2.50 |
| Phenylbenzimidazole sulfonic acid | 2.00 |
| Titanium dioxide | 3.00 |
| Mineral oil | 8.00 |
| Dicaprylyl ether | 7.00 |
| Butylene glycol dicaprylate/dicaprate | 4.00 |
| Phenylenemethyl polysiloxane | 2.00 |
| PVP hexadecene copolymer | 1.00 |
| Octoxyglycerin | 0.50 |
| Glycerin | 2.50 |
| Magnesium chloride | 0.70 |
| Tocopherol acetate | 1.00 |
| Polyol Soluble Licorice Extract P-U | 0.80 |
| Preservative(s), perfume | q.s. |
| Ethanol | 1.00 |
| Water | ad 100 |

### EXAMPLES OF W/O EMULSIONS

Example No.

| | 17 | 18 |
|---|---|---|
| Polyglyceryl-2-dipolyhydroxy stearate | 4.00 | 5.00 |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| Butylmethoxy dibenzoylmethane | 0.50 | 1.50 |
| 4-Methylbenzylidene camphor | 1.00 | 3.00 |
| Butylene glycol dicaprylate/dicaprate | 4.00 | 5.00 |
| Shea butter | — | 0.50 |
| Butylene glycol | — | 6.00 |
| Octoxyglycerin | — | 3.00 |
| Glycerin | 5.00 | — |
| Tocopherol acetate | 0.50 | 1.00 |
| Polyol Soluble Licorice Extract P-U | 0.2 | 0.1 |
| EDTA | 0.20 | 0.20 |
| Preservative(s) | q.s. | q.s. |
| Ethanol | — | 3.00 |
| Perfume | q.s. | q,s, |
| Water | ad 100 | ad 100 |

### EXAMPLE (W/O CREAM)

Example No. 19

| | |
|---|---|
| Polyglyceryl-3-diisostearate | 3.50 |
| Glycerin | 3.00 |
| Polyglyceryl-2-dipolyhydroxystearate | 3.50 |
| Polyol Soluble Licorice Extract P-U | 0.25 |
| Preservative(s) | q.s. |
| Perfume | q.s |
| Magnesium sulfate | 0.6 |
| Isopropyl stearate | 2.0 |
| Caprylyl ether | 8.0 |
| Cetearyl isononanoate | 6.0 |
| Water | ad 100 |

### EXAMPLE (W/O EMULSION)

Example No. 20

| | |
|---|---|
| Triceteareth-4-phosphate | 0.80 |
| Butylated hydroxytoluene | 0.05 |
| Glyceryl lanolate | 1.70 |
| Cyclomethicone | 2.20 |
| Isopropyl palmitate | 1.00 |
| Polyol Soluble Licorice Extract P-U | 0.050 |
| Polyacrylic Acid | 0.50 |
| Ethylenediamine tetraacetic acid | 1.00 |
| Sodium hydroxide | q.s. |
| Citric acid | 0.01 |
| Preservative(s) | q.s. |
| Perfume | q.s |
| Water | ad 100 |

1.-4. (canceled)

5. A method of treating or preventing symptoms of intrinsic or extrinsic skin ageing, wherein the method comprises applying a cosmetic or dermatological preparation comprising at least one of licochalcone A and an extract from Radix Glycyrrhizae inflatae that contains licochalcone A to skin.

6. The method of claim 5, wherein the preparation comprises from 0.0001% to 5% by weight of licochalcone A, relative to a total weight of the preparation.

7. The method of claim 6, wherein the preparation comprises from 0.001% to 1% by weight of licochalcone A.

8. The method of claim 6, wherein the preparation comprises from 0.005% to 0.15% by weight of licochalcone A.

9. The method of claim 6, wherein the preparation further comprises from 0.001% to 10% by weight of one or more polyols, relative to a total weight of the preparation.

10. The method of claim 7, wherein the preparation further comprises from 0.01% to 5% by weight of one or more polyols, relative to a total weight of the preparation.

11. The method of claim 8, wherein the preparation further comprises from 0.05% to 2% by weight of one or more polyols, relative to a total weight of the preparation.

12. The method of claim 9, wherein the one or more polyols comprise butylene glycol.

13. The method of claim 5, wherein the preparation comprises an O/W cream.

14. The method of claim 5, wherein the preparation comprises a W/O emulsion.

15. The method of claim 5, wherein at least one of the following ageing processes or conditions are at least one of treated and prevented:
deficient or hypoactive skin conditions or deficient or hypoactive conditions of skin appendages,
phenomena of premature ageing of at least one of skin and skin appendages,
negative changes in at least one of skin and skin appendages caused by the environment,
skin damage caused by light,
pigmentation disorders,
itching,
dry skin conditions and horny layer barrier disorders
hair loss.

16. A method of treating or preventing damaging effects of ultraviolet radiation on skin, wherein the method comprises applying a cosmetic or dermatological preparation comprising at least one of licochalcone A and an extract from Radix Glycyrrhizae inflatae that contains licochalcone A to skin.

17. The method of claim 16, wherein the preparation comprises from 0.0001% to 5% by weight of licochalcone A, relative to a total weight of the preparation.

18. The method of claim 17, wherein the preparation comprises from 0.001% to 1% by weight of licochalcone A.

19. The method of claim 17, wherein the preparation comprises from 0.005% to 0.15% by weight of licochalcone A.

20. The method of claim 17, wherein the preparation further comprises from 0.001% to 10% by weight of one or more polyols, relative to a total weight of the preparation.

21. The method of claim 18, wherein the preparation further comprises from 0.01% to 5% by weight of one or more polyols, relative to a total weight of the preparation.

22. The method of claim 19, wherein the preparation further comprises from 0.05% to 2% by weight of one or more polyols, relative to a total weight of the preparation.

23. The method of claim 20, wherein the one or more polyols comprise butylene glycol.

24. The method of claim 16, wherein the preparation comprises an O/W cream or a W/O emulsion.
